(19) United States
(12) Patent Application Publication
(10) Pub. No.: US 2011/0177142 A1
(43) Pub. Date: Jul. 21, 2011

(54) DISPERSION OF WAXES AND INORGANIC NANOPARTICLES AND USE THEREOF

(76) Inventors: Ulrich Nolte, Kleve (DE); Berkei Michael, Heltern am see (DE)

(21) Appl. No.: 12/989,176

(22) PCT Filed: Mar. 26, 2009
(86) PCT No.: PCT/EP2009/002220

§ 371 Date: Apr. 11, 2011

(30) Foreign Application Priority Data
Apr. 25, 2008 (DE) 102008021007.2

(51) Int. Cl. A61K 8/04; C09D 191/06; C09D 11/00; A61Q 3/02

(52) U.S. Cl. 424/401; 106/270; 106/31.13

(57) ABSTRACT

The invention relates to dispersions, wherein the dispersions—in addition to a dispersant—compromise in combination (a) on the hand particles based on at least one organic material containing or being made of at least on wax, in particular wax particles, and (b) on the other hand particles based on at least one inorganic material, in particular inorganic nanoparticles, and to the use of said dispersions.

### CROSS-REFERENCES TO RELATED APPLICATIONS

This application is a National Stage filing of International Application PCT/EP 2009/002220, filed Mar. 26, 2009, claiming priority to German Application No. DE 10 2008 021 007.2 filed Apr. 25, 2008, entitled “DISPERSION OF WAXES AND INORGANIC NANOPARTICLES AND USE THEREOF.” The subject application claims priority to PCT/EP 2009/002220, and to German Application No. DE 10 2008 021 007.2, and incorporates all by reference herein, in their entirety.

### BACKGROUND OF THE INVENTION

The present invention relates to dispersions which comprise in combination firstly particles based on at least one organic material, which comprises or consists of at least one wax, in particular wax particles, and secondly particles based on at least one inorganic material, in particular inorganic nanoparticles, and also to the use of these dispersions, in particular as and/or in coating materials and coating systems, such as, in particular, paints, inks, and the like, in plastics, in foams, as or in cosmetics, in particular nail varnishes, as or in adhesives, as or in sealants, etc.

In particular the present invention relates to dispersions comprising waxes firstly and inorganic nanoparticles secondly, and also to the use thereof, as described above.

Additionally the present invention relates equally to systems, in particular coating materials and coating systems such as, in particular, paints, inks, and the like, plastics, foams, and cosmetics, such as, in particular, nail varnishes, which comprise these dispersions.

Finally the present invention relates to the use of a combination of particles based on at least one organic material, which comprises or consists of at least one wax, in particular wax particles, firstly and particles based on at least one inorganic material, in particular inorganic nanoparticles, secondly in coating materials and coating systems, in particular paints, inks, and the like, in dispersions of all kinds, in plastics, in foams, in cosmetics, in particular nail varnishes, in adhesives, and in sealants.

For improving the mechanical properties of coating systems and dispersion systems (as, for example, of paints, inks such as printing inks, coatings) and of plastics, in particular, specifically, for improving their wear properties, such as scratch resistance and abrasion resistance, the incorporation of additives and fillers is known in principle to the skilled person.

While in certain circumstances the filler particles known from the prior art do improve the mechanical properties, such as the scratch resistance and/or abrasion resistance, for example, of the relevant coating systems (e.g., of paints) in which they are used, there may nevertheless, following application, be increased embrittlement in the resultant coating film (e.g., in a paint film). Additionally, the incorporation of these filler particles often results in unwanted clouding and lack of transparency of the coating system. Additionally, relatively high filler levels are frequently necessary in order to achieve the desired effects, and this makes it more difficult to stabilize the resultant dispersion systems and is also unwanted on cost grounds.

WO 2007/072189 A2 relates to a silylated polymer emulsion further comprising nanosilicate, and to its use for coating systems. With the emulsions described therein, however, it is not always possible to obtain the desired performance properties.

EP 0 960 871 A2 relates to aqueous preparations for the treatment of mineral construction materials, the aqueous preparations comprising, in addition to an emulsion of a polyfunctional carbosilane and/or condensation product thereof, an emulsion of an organopolysiloxane, and a water-dispersible or water-emulsifiable organic polymer, inorganic nanoparticles as well.

JP 07138484 A relates to the production of extrudates from a mixture of wax, oils or resins and a pulverulent inorganic material, such as talc or silica, for example. The incorporated additional components are said to have effects including improved fluidity in the extrusion process with the wax.

### BRIEF SUMMARY OF THE INVENTION

The present invention is now based on the object of providing filler particles of the abovementioned kind, and disperse systems, in particular dispersions, comprising these filler particles, which are suitable in particular for use in the aforementioned systems and which at least largely avoid, or else at least attenuate, the disadvantages associated with the conventional particles.

A further object of the present invention lies in the provision of innovative disperse systems, in particular dispersions, which comprise filler particles of the type specified at the outset and which, when incorporated into the systems specified at the outset, produce an efficient performance boost and are suitable in particular for improving the mechanical properties of coating systems (as, for example, of paints, inks, such as printing inks, coatings, etc.) and of plastics, in particular, specifically, for increasing their wear properties, in particular the scratch resistance and abrasion resistance, but in particular without at the same time adversely affecting other required performance properties of these systems (such as, for example, gloss characteristics, surface smoothness, adhesion, etc.).

For the solution of the problem outlined above, the present invention therefore proposes dispersions as disclosed herein.

The present invention further provides for the use of the dispersions of the invention in coating materials and coating systems, in particular paints, inks, and the like, in dispersions of all kinds, in plastics, in foams, in cosmetics, in particular nail varnishes, in adhesives, and in sealants, as shown herein.

Further provided, in turn, by the present invention are coating materials and coating systems, in particular paints, inks, and the like, plastics, foams, cosmetics, in particular nail varnishes, adhesives, and sealants, which comprise the dispersions according to the present invention as disclosed herein.

Further provided by the invention, finally, is the use of a combination of particles based on at least one organic material, which comprises or consists of at least one wax, in particular wax particles, firstly and particles based on at least one inorganic material, in particular inorganic nanoparticles, secondly in coating materials and coating systems, in particular paints, inks, and the like, in dispersions of all kinds, in plastics, in foams, in cosmetics, in particular nail varnishes, in adhesives, and in sealants as disclosed herein.

### DETAILED DESCRIPTION OF THE INVENTION

It will be appreciated that, in the text below, those statements made only in relation to an individual aspect of the present invention also apply equally, correspondingly, to the other aspects of the present invention, without need for this to be explicitly mentioned.

The present invention accordingly provides—in accordance with a first aspect of the present invention—dispersions, where the dispersions comprise—as well as a dispersion medium—in combination
(a) firstly particles based on at least one organic material which comprises or consists of at least one wax, in particular wax particles, and
(b) secondly particles based on at least one inorganic material, in particular inorganic nanoparticles.

A particular feature of the present invention should therefore be seen, in particular, as being the fact that, in the context of the present invention, (a) particles based on at least one organic material, which comprises or consists of at least one wax, in particular wax particles, firstly and (b) particles based on at least one inorganic material, in particular inorganic nanoparticles secondly, are united in combination in a single dispersion, which is then suitable in particular for use in coating materials and coating systems, in particular paints, inks or the like, in dispersions of all kinds, in plastics, in foams, in cosmetics, in particular nail varnishes, in adhesives, and also in sealants, and in particular improves their mechanical properties.

As the applicant has surprisingly found out, it becomes possible in this way, in particular, to reduce significantly, as compared with the respective individual systems, the amounts of the above-stated ingredients and/or additives (i.e., wax-containing organic particles firstly and inorganic particles secondly) that are needed for improving, in particular, mechanical properties of coatings, such as scratch resistance and abrasion resistance, for example, and this fact suggests a synergism between the aforementioned wax materials firstly and the inorganic materials, in particular nanoparticles, secondly.

The combination of the two aforementioned components (a) and (b) improves in particular the long-term stability of surfaces with respect to mechanical damage.

The invention makes it possible, for example, to incorporate inorganic particles, in particular inorganic nanoparticles, in fine division in waxes, subsequently into high-melting polymer melts which at the same time are generally of high viscosity, such as thermoplastics or powder coatings, for example, in a simple way.

The combination of inorganic particles, in particular inorganic nanoparticles, with waxes may in particular also mean that wax particles coated with nanoparticles diffuse to the surfaces of initially liquid and then solid polymer matrices and positively modify or improve these matrices at the interface. For wax particles as such, this is a long-established phenomenon for the skilled person.

The technology according to the invention can be employed for the application of nanomaterials in solid matrices based on fusible waxes.

The above-outlined synergism between wax particles firstly and inorganic particles, in particular nanoparticles, secondly may therefore be efficiently exploited for improving the properties, for example, of paints and plastics.

The present invention accordingly provides disperse inorganic-organic hybrid systems with a particulate, wax-based organic material firstly and a particulate, in particular nanoparticulate, inorganic material secondly.

The dispersions of the invention, as a result, unite the positive properties of the waxes firstly and of the relevant inorganic material, in particular of inorganic nanoparticles, secondly in a single system and, when incorporated into coating materials and coating systems of the aforementioned type, result in a significant improvement in their mechanical properties, in particular in an increase in the wear resistance, in particular the scratch resistance and/or the abrasion resistance, and do so while substantially retaining or, under certain circumstances, even improving the other required performance properties (such as, for example, surface smoothness, gloss, etc.). Moreover, the dispersions of the invention can be incorporated homogeneously and stably into the aforementioned systems. Their incorporation into the aforementioned systems, in particular into coating materials and coating systems, such as paints, inks, and the like, leads in particular to no clouding at all of the systems in question.

The applicant has now surprisingly found out that partitularly good results can be obtained when (a) the organic particles, in particular wax particles, firstly and (b) the inorganic particles, in particular inorganic nanoparticles, secondly have a bimodal particle size distribution.

It has turned out to be particularly advantageous if the ratio V of the average particle diameter of the particles (a), Ø_{particle (a)}, to the average particle diameter of the particles (b), Ø_{particle (b)}, is governed by:

$$V = \frac{{∅}_{particle ( a )}}{{∅}_{particle ( b )}} > 1 ,$$

in particular >2, preferably >3, very preferably >5.

It is especially advantageous if the ratio V of the average particle diameter of the particles (a), Ø_{particle (a)}, to the average particle diameter of the particles (b), Ø_{particle (b)}, varies in the range from 1.05 to 1000, in particular 2.05 to 500, preferably 3.05 to 250, very preferably 5.05 to 100.

As far as the organic particles (a), in particular wax particles, present in the dispersions of the invention are concerned, their particle size may vary within wide ranges. Generally speaking, the organic particles (a), in particular wax particles, have particle sizes in the range from 5 nm to 500 μm, in particular 10 nm to 200 μm, preferably 20 nm to 150 μm, more preferably 30 nm to 125 μm, very preferably 50 nm to 100 μm. The particle sizes may be determined, for example, by means of transmission electron microscopy, analytical ultracentrifugation, or by methods of light scattering.

As far as the inorganic particles (b) present in the dispersions of the invention are concerned, their particle size may also vary within wide ranges. Generally speaking, the inorganic particles (b) have particle sizes in the range from 0.5 to 1000 nm, in particular 2 to 800 nm, preferably 5 to 700 nm, more preferably 10 to 600 nm, very preferably 15 to 500 nm, especially preferably 20 to 100 nm. As described above, the particle sizes may be determined, for example, by means of transmission electron microscopy, analytical ultracentrifugation, or by methods of light scattering.

It will be appreciated that, for all of the size and range indications given in the context of the present invention, it may be necessary, in certain cases or on an application basis, to deviate from the specified figures, without departing from the scope of the present invention.

Furthermore, the applicant has now surprisingly found out that particularly good results can be obtained when, in the dispersions of the invention, the weight-based ratio M of the particles (b) to the particles (a) is at least 0.01, in particular at least 0.5, preferably at least 1, very preferably at least 2.

More preferably, in the dispersions of the invention, the weight-based ratio M of the particles (b) to the particles (a) varies in the range from 0.01 to 500, in particular 0.5 to 250, preferably 1 to 150, very preferably 2 to 100.

As far as the amount of organic particles (a), in particular wax particles, present in the dispersions of the invention is concerned, this amount may vary within wide ranges. Generally speaking, the dispersions of the invention contain the wax-containing particles (a), based on the dispersions, in amounts of 0.01% to 30%, in particular 0.05% to 20%, preferably 0.1% to 15%, very preferably 0.1% to 10%, especially preferably 0.2% to 5% by weight.

As far as the amount of inorganic particles (b) present in the dispersions of the invention is concerned, this amount may also vary within wide ranges. Generally speaking, the dispersions of the invention contain the inorganic particles (b), based on the dispersions, in amounts of 0.01% to 50%, in particular 0.1% to 40%, preferably 0.2% to 30%, very preferably 0.3% to 20%, especially preferably 0.5% to 10% by weight.

As far as the inorganically based material of the particles (b) is concerned, it may be formed of at least one optionally doped inorganic oxide (e.g., TiO₂, ZnO, Al₂O₃, SiO₂, CeO₂, Fe₂O₃, Fe₃O₄, etc.), hydroxide (e.g., Al[OH]₃ etc.), oxide hydroxide (e.g., AlOOH etc.), sulfate (e.g., alkaline earth metal sulfates, such as barium sulfate, calcium sulfate, etc.), phosphate (e.g., alkaline earth metal phosphate, such as calcium phosphate, or lanthanum phosphate, etc.), sulfide (e.g., cadmium sulfide, zinc sulfide, etc.), selenide, telluride, vanadate (e.g., BiVO₄ or YVO₄:Eu³⁺), tungstate (e.g., CaWO₄ or Yb₂(WO₄)₃), titanate (e.g., BaTiO₃), fluoride (e.g., CaF₂, CaF₂:Eu²⁺, NaYF₄ or YbF₃:Yb³⁺,Er³⁺), apatite, borate, carbonate (e.g., alkaline-earth metal carbonate, such as magnesium carbonate or calcium carbonate, etc.), nitride (e.g., AlN, Si₃N₄, etc.), silicate (e.g., alkaline earth metal silicate, such as calcium silicate, layer silicates and phyllosilicates, etc.), zeolite, carbide (e.g., SiC etc.), single-wall or multiwall carbon nanotubes and/or metal/element (e.g., silver, copper, fullerene), intermetallic phases, or else of mixtures or combinations of such compounds, or may comprise said compound(s).

Advantageously, the above-stated inorganically based material of the particles (b) from the group of optionally doped inorganic oxides, hydroxides, oxide hydroxyides, sulfates, phosphates, sulfides, selenides, tellurides, fluorides, apatites, zeolites, carbonates, nitrides, silicates, carbides and/or metals/elements is of low solubility in the respective medium.

In particular the inorganically based material of the particles (b) may be formed of at least one optionally doped oxide, hydroxide, oxide hydroxide, sulfate, phosphate, sulfide, selenide, telluride, fluoride, apatite, carbonate, nitride, silicate, carbide of at least one metal or semimetal, or else of zeolites or else of metals or else of mixtures or combinations of such compounds, or may comprise said compound(s).

Preferably, the inorganically based material of the inorganic particles (b) may be formed of at least one optionally doped oxide, hydroxide and/or oxide hydroxide of aluminum, silicon, zinc, titanium, cerium and/or iron, an optionally doped alkaline earth metal sulfate, an optionally doped alkaline earth metal phosphate, transition metal phosphate or lanthanoid phosphate, an optionally doped alkali metal vanadate, alkaline earth metal vanadate, transition metal vanadate or lanthanoid vanadate, an optionally doped alkali metal fluoride, alkaline earth metal fluoride, transition metal fluoride or lanthanoid fluoride, a cadmium sulfide or zinc sulfide, a zinc selenide or cadmium selenide, a cadmium telluride or bismuth telluride, an optionally doped alkaline earth metal carbonate, an aluminum nitride or silicon nitride, an optionally doped alkaline earth metal silicate, a silicon carbide, silver, carbon allotropes or else of mixtures or combinations of such compounds, or may comprise said compound(s).

Particularly preferred for forming the inorganically based material of the inorganic particles (b) are the following compounds: Ti_{O2}, ZnO, Al₂O₃, SiO₂, CeO₂, Fe₂O₃, Fe₃O₄, Al(OH)₃, Al(O)OH, alkaline earth metal sulfates (e.g., barium sulfate, calcium sulfate, etc.), alkaline earth metal phosphates (e.g., calcium phosphate), optionally doped YVO₄, NaYF₄:Yb₃Er, optionally doped YbF, optionally doped CaF₂, optionally doped cerium phosphate, optionally doped lanthanum phosphate, zinc phosphate, barium titanate, antimony tin oxide, indium tin oxide, copper oxide, optionally doped CaWO₄, optionally doped Yb₂(WO₄)₃, optionally doped Y₂(WO₄)₃, cadmium sulfide, zinc sulfide, alkaline earth metal carbonate (e.g., magnesium carbonate, calcium carbonate, etc.), AlN, Si₃N₄, alkaline earth metal silicates (e.g., calcium silicate etc.), SiC, carbon nanotubes and/or silver, and also mixtures or combinations of such compounds.

It is especially preferred if the inorganically based material of the inorganic particles (b) is formed of aluminum oxide, silicon dioxide, zinc oxide, cerium oxide, boehmite and/or titanium dioxide, or comprises said compound(s).

In accordance with one particular embodiment, provision may be made for the inorganically based material of the inorganic particles (b) to be of surface-modified design, in which case such surface modification takes place advantageously by means of polysiloxane groups; in other words, with this embodiment, polysiloxane groups are applied at or on the surface of the inorganically based material of the inorganic particles (b), preferably by means of physical and/or chemical, in particular chemical covalent, bonding.

The effect of such surface modification by means of polysiloxane groups is an even greater increase or improvement in the performance properties of the dispersions of the invention, in particular when they are incorporated into coating materials and coating systems. The surface modification, preferably with polysiloxane groups, leads in particular, to a reduced tendency toward sedimentation and toward gel formation on the part of the dispersions of the invention. Additionally, embrittlement of the dried and/or cured coating system is efficiently counteracted. A further advantage of the surface modification is that, on incorporation into coating systems, the interaction with the binder is advantageously influenced and, in this way, transparency and refractive index are improved still further in relation to non-surface-modified particles, with significantly lower light scattering occurring in particular because of the reduced difference in refractive index.

The surface modification, in particular by means of polysiloxane groups, is known in principle to the skilled person from the prior art. In this respect, reference may be made to the patent applications DE 10 2005 006 870 A1 or EP 1 690 902 A2, and DE 10 2007 030 285 A1 or PCT/EP 2007/006273, which originate from the applicant itself, and whose entire disclosure content is hereby incorporated by reference. All of the aforementioned publications relate to the surface modification of metal- or semimetal-oxidic or hydroxidic surfaces by means of polysiloxanes, advantageously through formation of chemical, in particular covalent, bonds.

As far as the organically based material of the wax-based organic particles (a) is concerned, this organically based material is formed of at least one wax or comprises such a wax. The wax may in particular be selected from the group of (i) natural waxes, in particular plant, animal, and mineral waxes; (ii) chemically modified waxes; (iii) synthetic waxes; and also mixtures thereof.

In accordance with one particularly preferred embodiment of the present invention, organically based material used in the wax-based organic particles (a) is a synthetic wax, in particular a polyolefin-based wax, preferably a wax based on an oxidized polyolefin. As far as the concept of the wax is concerned, the term constitutes a phenomenological designation for a series of substances which are obtained naturally or artificially or synthetically and which in general have the following properties: waxes are kneadable at 20° C., solid to brittly hard, coarsely to finely crystalline, translucent to opaque, but not glasslike, melting without decomposition above 40° C., but, at just a little above the melting point, being of relatively low viscosity and generally, and advantageously, being non-stringing; they have a highly temperature-dependent consistency and solubility, and can be polished under gentle pressure. If more than one of the qualities listed above is absent, this substance, according to the DGF (Deutsche Gesellschaft fur Fettwissenschaften (German Society for Fat Sciences)), is not a wax (cf. DGF Standard Method M-I 1 (75)).

Waxes differ from similar synthetic or natural products (e.g., resins, plastic masses, metal soaps, etc.) primarily in that in general at about between 50 and 90° C., in exceptional cases also up to about 200° C., they undergo transition to the low-viscosity, liquid-melt state and are virtually free from ash-forming compounds.

Waxes form pastes or gels and burn in general with a sooty flame.

According to their origin, the waxes are divided into three groups, namely (i) natural waxes including plant waxes (e.g. candelilla wax, carnauba wax, japan wax, espartograss wax, cork wax, guaruma wax, rice germ oil wax, sugar cane wax, ouricury wax, montan wax, etc.), animal waxes (e.g., beeswax, shellac wax, spermaceti, lanolin or wool wax, uropygial grease, etc.), and mineral waxes (e.g., ceresin, ozokerite or earth wax, etc.); (ii) chemically modified waxes, including hard waxes (e.g., montan ester waxes, Sasol waxes, hydrogenated jojoba waxes, etc.); and (iii) synthetic waxes, including polyalkylene waxes, polyalkylene glycol waxes (e.g., polyethylene glycol waxes), etc.

The major constituent of natural recent (“renewable”) waxes are esters of long-chain fatty acids (wax acids) with long-chain fatty alcohols, triterpene alcohols or steroid alcohols; these wax esters also contain free carboxyl and/or hydroxyl groups, which give the so-called wax soaps their emulsifying capacity. Natural fossil waxes, from lignite or petroleum, for example, consist primarily—like waxes from the Fischer-Tropsch synthesis or polyalkylene waxes (e.g., polyethylene waxes)—of straight-chain hydrocarbons; the former, however, depending on provenance, may also include branched or cycloaliphatic hydrocarbons. Frequently these “hydrocarbon” waxes are functionalized by subsequent oxidation or else, in the case of the polyolefin waxes, by comonomers having carboxyl groups.

For further details on the concept of waxes, reference may be made, for example, to Römpp Chemielexikon, 10th edition, volume 6, 1999, Georg Thieme Verlag Stuttgart/New York, page 4906, entry heading: “Wachse” [Waxes] and also to the references cited therein, especially Cosm. Toil. 101, 49 (1986), and also DGF Einheitsmethoden [Standard methods], department M-Waxes and Wax Products, 7th supplement 05/1999, Stuttgart: Wissenschaftliche Verlagsgesellschaft, the aforementioned literature references being hereby incorporated in their entirety into the present specification by reference.

In accordance with the invention it is preferred if, as organically based material of the wax-based organic particles (a), a wax is used which contains functional groups. The functional groups are preferably polar groups, in particular groups which contain heteroatoms from the group of O, N and/or S, preferably O, preferably hydroxyl groups, polyether groups, in particular polyalkylene oxide groups, and/or carboxyl groups, very preferably polyether groups and/or hydroxyl groups. The functional groups of these waxes result in improved or more stable adhesion.

In principle the dispersions of the invention may be formed on an aqueous basis and/or comprise water as dispersion medium.

In addition, however, there also exists in principle the possibility for the dispersions of the invention to be formed on an organic basis and/or to comprise at least one organic solvent as dispersion medium.

In addition, the dispersions according to the present invention further comprise at least one further ingredient and/or at least one additive, which may be in particular selected from the group of emulsifiers, wetting agents, antioxidants, stabilizers, neutralizing agents, thickeners, dispersants, organic solvents, solubilizers, and biocides, and also mixtures thereof.

The dispersions according to the present invention may be formed or present as coating compositions or coating systems, in particular paints, inks or the like, as cosmetics, in particular nail varnishes, as adhesives, and as sealants.

Equally, the dispersions according to the present invention may be designed for incorporation into coating materials and coating systems, in particular paints, inks or the like, into plastics, into foams, into cosmetics, in particular nail varnishes, into adhesives, and into sealants. Typically for this purpose the dispersions according to the present invention are used in amounts of 0.1% to 30%, in particular 0.5% to 20%, preferably 1% to 10%, by weight, based on the resulting overall system.

As far as the process for preparing the dispersions according to the present invention is concerned, it is possible in accordance with the invention to adopt a procedure as follows: for preparing the dispersions of the invention it is possible, for example, for melted waxes, wax dispersions or wax emulsions with inorganic particles, in particular inorganic nanoparticles, in the form, for example, of inorganic particle dispersions in water or organic solvents, to be mixed and then added in their combination, in the form of the dispersion, so to speak, as an already fully combined additive, to—for example—a paint, a printing ink, a plastic or the like, or else alternatively, in these applications, to be mixed in in situ, i.e., used as a combination of two additives. In this case, the combination of inorganic nanoparticles firstly and wax particles secondly advantageously has a bimodal particle size distribution, comprising a larger organic material in combination with a more finely divided inorganic material, as described above. The division or mixing or homogenization of inorganic particles, in particular nanoparticles, with waxes may take place, for example, by introduction of the necessary energy, in particular by simple, e.g., low-shear-force stirring or mixing, but also, in particular, by using dissolvers, Dispermats, bead mills and ball mills, ultrasound, etc., optionally with additional use of wetting and dispersing additives, as described above. Whereas the particle size of the inorganic particles is determined generally by the starting material used, the particle size of the wax-based particles may be determined either through selection of the starting material used (e.g., wax dispersions or wax emulsions or micronized waxes) or else adjusted in a targeted way through the energy introduced (e.g., shear forces, etc.). Suitable starting materials are commercially customary and are available to the skilled person in sufficient selection and variability.

For further details relating to the preparation of the dispersions of the invention, it is possible, in order to avoid unnecessary repetition, to refer to the above statements concerning the dispersions of the invention, which apply equally in relation to the preparation process, and to refer to the working examples later on below.

Further provided by the present invention—in accordance with a second aspect of the present invention—is the use of the dispersions of the invention in coating materials and coating systems, in particular paints, inks, and the like, in dispersions of all kinds, in plastics, in foams, in cosmetics, in particular nail varnishes, in adhesives, and in sealants.

The dispersions of the invention may be used in particular for contributing to improving the mechanical properties, in particular to increasing the wear resistance, preferably the scratch resistance and/or abrasion resistance, in the aforementioned systems.

Further provided by the present invention—in accordance with a third aspect of the present invention—are coating materials and coating systems, in particular paints, inks, and the like, plastics, foams, cosmetics, in particular nail varnishes, adhesives, and sealants, which comprise the dispersions of the invention.

Typically, in accordance with this aspect of the invention, the dispersions according to the present invention are used in amounts of 0.1% to 30%, in particular 0.5% to 20%, preferably 1% to 10%, by weight, based on the resulting overall system.

Further provided by the present invention—in accordance with a fourth aspect of the present invention—is the use of a combination of
(a) particles based on at least one organic material, which comprises or consists of at least one wax, in particular wax particles, firstly and
(b) particles based on at least one inorganic material, in particular inorganic nanoparticles, secondly
   in coating materials and coating systems, in particular paints, inks, and the like, in dispersions of all kinds, in plastics, in foams, in cosmetics, in particular nail varnishes, in adhesives, and in sealants.

The combination of the invention may be used in particular for contributing to improving the mechanical properties, in particular to increasing the wear resistance, preferably the scratch resistance and/or abrasion resistance, in the aforementioned systems.

With the dispersions of the invention, organically-inorganically based hybrid systems based on organic wax particles firstly and on inorganic particles, in particular inorganic nanoparticles, secondly are provided which, when incorporated into the aforementioned systems, result in a significant performance boost, in particular in a significant improvement in the mechanical properties, in particular the wear resistance, preferably the scratch resistance and/or abrasion resistance. The joint incorporation of organic wax-particles firstly and inorganic particles, in particular inorganic nanoparticles, secondly produces a synergistic intereaction such that, relative to use alone, the relevant amounts for obtaining the same effect can be significantly reduced or an increased effect produced with the same amounts.

By means of the present invention, therefore, success has been achieved in uniting the performance advantages of inorganically based nanoparticles firstly and wax-containing particles secondly in a single dispersion. In particular, when incorporated into the aforementioned systems (i.e., coating materials and coating systems, in particular paints, inks or the like, plastics, foams, cosmetics, in particular nail varnishes, adhesives, and sealants), the dispersions of the invention produce an efficient performance boost and are suitable in particular for improving the mechanical properties of said systems, in particular, their wear properties, such as scratch resistance and abrasion resistance, but in particular without adversely affecting other required performance properties of these systems (e.g., gloss characteristics, surface smoothness, adhesion capacity, etc.).

In this way, effective fillers are provided for the aforementioned systems, which, on incorporation, for example, into coating systems (e.g., paints and inks), efficiently improve the mechanical properties of the coating, such as abrasion resistance and scratch resistance, for example.

In the context of the present invention, as a result, (a) particles based on at least one organic material, which consists of or comprises at least one wax, in particular wax particles, firstly and (b) particles based on at least one inorganic material, in particular inorganic nanoparticles, secondly, are united in combination in a single dispersion, which is then suitable in particular for use in coating materials and coating systems, in particular paints, inks or the like, and dispersions of all kinds, in plastics, in foams, in cosmetics, in particular nail varnishes, in adhesives, and also in sealants, and in particular improves their mechanical properties. In this way, surprisingly, it becomes possible in particular to achieve significant reductions in the amounts of the aforementioned ingredients or additives (i.e., wax-containing organic particles firstly and inorganic particles secondly) that are needed to improve, in particular, mechanical properties of coatings, such as scratch resistance and abrasion resistance, for example, relative to the respective individual systems, and this can be attributed to a synergism between the aforementioned wax materials firstly and the inorganic particles, in particular nanoparticles, secondly.

The above-outlined synergism between wax particles firstly and inorganic particles, in particular nanoparticles, secondly can therefore be efficiently exploited for improving the properties, for example, of paints and plastics.

As described above, the dispersions of the invention improve the mechanical properties of coatings in comparison to pure wax emulsions or to pure inorganic nanoparticle dispersions, in a significant way.

In comparison to the individual constituents, i.e., wax particles firstly and inorganic nanoparticles secondly, the dispersions of the invention display a synergistic effect, in particular in relation to the mechanical properties of the coatings, meaning that the total amount of particles to be incorporated can be reduced significantly in comparison to the pure materials, in particular in comparison to pure inorganic nanoparticle dispersions.

The possible applications of the dispersions of the invention are extremely broad. This broad usefulness in combination with the extremely good efficiency of the dispersions of the invention far exceed dispersions of the prior art.

The dispersions of the invention may be employed, for example, by addition to existing systems which are processed further to form, for example, paints, adhesives, plastics, etc. The addition even of small amounts of the dispersions of the invention produces an extraordinarily increased mechanical resistance. Surprisingly, the other processing properties of the systems in question, in particular paints, plastics, etc., are influenced not at all or not significantly, and so there is no need for new optimization of the other parameters in the case of these applications.

The dispersions of the invention are therefore outstandingly suitable for use in coating materials of all kinds, plastics, adhesives, sealants, etc.

Further embodiments, modifications, and variations of the present invention are readily discernible and realizable for the skilled person from a reading of the description, without departing from the scope of the present invention.

The present invention is illustrated via the working examples below, which, however, are not intended in any way to restrict the present invention.

### WORKING EXAMPLES

### Preparation of Inventive Dispersions

### Example 1

### Preparation of an Aqueous Nanoparticle Dispersion

In 174 g of water, 6 g of a polymeric wetting and dispersing additive with groups having pigment affinity are mixed in. Added to this mixture with stirring are 120 g of a commercial aluminum oxide nanopowder having a primary particle size of d50=40 nm. This mixture is then dispersed, following addition of 250 ml of glass beads (diameter 1 mm), by means of a Dispermat at approximately 2000 rpm for 3 hours. The glass pearls are then removed by sieving and the dispersion thus obtained is ready.

### Example 2

### Preparation of a Solvent-Based Nanoparticle Dispersion

In 262 g of nonpolar solvent (Exxsol D60), 18 g of a polymeric wetting and dispersing additive with groups having, pigment affinity are dissolved. Added to this mixture with stirring are 120 g of a commercial boehmite having a primary particle size of d50=10 nm. This mixture is then dispersed by means of ultrasound for 2 hours. The dispersion thus obtained is stable for a number of months.

### Example 3

### Preparation of a Wax Emulsion

In a pressure-rated apparatus, 150 g of water, in which 1.3 g of a low molecular weight anionic surfactant have been dissolved, are admixed slowly with 50 g of a melted, oxidized PE wax, using high shear forces by means, for example, of a dissolver disk, to form a stable oil-in-water emulsion. The final size of the wax particles in the dispersion (because of the preparation process, also called emulsion) droplet size is determined by light scattering as d50=50 nm.

### Example 4

### Preparation of a Wax Dispersion

In a pressure-rated apparatus, 60 g of a carnauba wax are dissolved in 320 g of solvent naphtha, with heating. Subsequently, 80 g of n-butanol are added to the mixture with intensive stirring. The wax, which is insoluble in this mixture, precipitates in the form of wax particles having a size (d50) of 10 μm. The dispersion thus obtained is stable for a number of months.

### Example 5

### Preparation of a Nano-Wax Premix

Added to the nanodispersion prepared in example 1, in a dissolver (2000 rpm) and using a toothed disk, continuously, is the required amount to achieve the desired wax/nano-aluminum oxide ratio and dispersion 3. Following complete addition, the mixture is homogenized for a further 20 minutes.

Using the methods described above, dispersions of the invention were prepared with varying amounts of wax particles and inorganic nanoparticles.

The applicant has surprisingly found that it is particularly advantageous to mix the dispersions thus obtained with inorganic nanoparticles and the dispersions or emulsions with wax particles before adding them to the subsequent coating materials of any kind, plastics, adhesives, sealants, etc. On mixing, a combination of wax particles and inorganic nanoparticles occurs, and contributes to the synergistic effects already mentioned.

The dispersions obtained in this way were subsequently incorporated into a two-component PU coating system and then tested for their performance properties in respect of the coating, in particular gloss, scratch resistance, and lubricity. Relative to a pure wax emulsion or else to a pure nanoparticle dispersion, the dispersions of the invention exhibit, in particular, improved scratch resistance and abrasion resistance. The results are summarized in the table hereinafter.

### Performance Tests:

Except for the control sample and the samples coated with the pure wax, all of the other samples comprise the dispersions of the invention in the coating composition.

The scratch resistance was determined by the Satra test method after 500, 1000, 2000, and 4000 cycles, the evaluation of the resulting abrasion resistance taking place in accordance with a school grade system with evaluation grades from 1 to 5 (1=very good to 5=deficient). For this purpose, the coatings were each applied in the same coat thickness, and were caused to dry and cure under the same conditions for 24 hours. Thereafter, after a further 3 days of storage, the Satra scratch resistance test is carried out under conditions which are known per se for the skilled person, by means of an abrasion disk which rotates on the surface of the coatings, with the cycles stated above.

The gloss value was determined in accordance with DIN EN 67530, corresponding to ISO 2813, at an angle of 60°.

The surface smoothness (“slip”) was measured by measurement of the slip properties, via the percentage reduction in slip resistance. In this measurement method, the frictional force of a defined article on the coating surface is measured; in this case, a 500 g weight with a defined felt underlay is advanced by a tensile machine over the coating surface at a constant speed. The force required to achieve this is measured using an electronic force transducer. Relative to a control sample, the reduction in the force required is calculated in % in comparison to the control sample, from the force values. In the case of positive values, the sample is smoother than the control, and, in the case of negative values, the sample is rougher than the control.

The abrasion resistance was determined in accordance with the Taber abrasion method of ASTM D 4060 (statement of the weight loss) under a load of 500 g. The parameter stated is the loss in mass in mg.

Reproduced hereinafter is the table with the test results in an aqueous, air-drying polyurethane coating material.

| | | | | | | | Taber |
|---|---|---|---|---|---|---|---|
| | Scratch resistance | | | | | | abrasion |
| | (Satra) | | | | | Surface | resistance |
| | 500 | 1000 | 2000 | 4000 | Gloss | smoothness | (% |
| | cycles | cycles | cycles | cycles | (60°) | (“slip”) | hazing) |
| Control | 1 | 3 | 5 | 5 | 95 | — | 12.2 |
| Pure Al₂O₃ | 1 | 2 | 4 | 5 | 95 | −8% | 11.9 |
| dispersion | | | | | | | |
| from example | | | | | | | |
| 1 (Ø = | | | | | | | |
| 40 nm) 0.5% | | | | | | | |
| by wt. | | | | | | | |
| (comparative) | | | | | | | |
| Pure Al₂O₃ | 1 | 2 | 3 | 5 | 93 | −13% | 11.4 |
| dispersion | | | | | | | |
| from example | | | | | | | |
| 1 (Ø = | | | | | | | |
| 40 nm) 1% | | | | | | | |
| by wt. | | | | | | | |
| (comparative) | | | | | | | |
| Pure wax | 1 | 3 | 4 | 5 | 85 | +12% | 6.8 |
| emulsion | | | | | | | |
| from example | | | | | | | |
| 3 0.5% | | | | | | | |
| wax (comparative) | | | | | | | |
| Ex. 1: | 1 | 1 | 2 | 2-3 | 86 | −2% | 3.9 |
| Al₂O₃ (Ø = | | | | | | | |
| 40 nm) 0.5% | | | | | | | |
| by wt. + | | | | | | | |
| 0.5% wax | | | | | | | |
| emulsion | | | | | | | |
| from example | | | | | | | |
| 3 (inventive) | | | | | | | |
| Ex. 1: | 1 | 1 | 1 | 2 | 83 | −7% | 3.6 |
| Al₂O₃ (Ø = | | | | | | | |
| 40 nm) 1.0% | | | | | | | |
| by wt. + | | | | | | | |
| 0.5% wax | | | | | | | |
| emulsion | | | | | | | |
| from example | | | | | | | |
| 3 (inventive) | | | | | | | |

The weight percentages are based on the amount of the corresponding type of particle in the total solids of the coating system used.

The results above show that through the incorporation of the dispersions of the invention it is possible to achieve a significant improvement in the abrasion resistance and scratching resistance.

The above tests forcefully demonstrate the increased performance of the systems of the invention.

### Influence of the Bimodal Particle Size Distribution on the Performance of the Dispersion of the Invention

An investigation was carried out into the influence of the bimodal particle size distribution in relation to the performance of the dispersions of the invention. For this purpose, dispersions of the invention with different ratios V of the average particle diameter of the wax-based particles firstly and of the inorganic nanoparticles secondly were incorporated into the above-described, polyurethane-based coating system (amounts in each case: 1.0% by weight nanoparticles (Al₂O₃ nanoparticles with average particle diameter of approximately 40 nm and 0.5% by weight wax particles), the values selected for V being about 1.05, about 3.1, about 5.2, and about 10.1.

The best results are achieved with high V values. The results are summarized in the table hereinafter.

Shown hereinafter is the table with the test results.

| | Scratch resistance (Satra) | | | | |
|---|---|---|---|---|---|
| | 500 | 1000 | 2000 | 4000 | Taber abrasion |
| | cycles | cycles | cycles | cycles | resistance (mg) |
| V = 1.05 | 1-2 | 2-3 | 2-3 | 3 | 5.8 |
| V = 3.1 | 1-2 | 2 | 2-3 | 2-3 | 5.2 |
| V = 5.2 | 1 | 1-2 | 2 | 2-3 | 4.9 |
| V = 10.1 | 1 | 1-2 | 1-2 | 2 | 3.8 |

### Influence of the Proportion on the Performance of the Dispersions of the Invention

An investigation was carried out into the influence of the proportion M of inorganic nanoparticles firstly and wax-based particles secondly, operating with varying values for M (M=about 0.6 or about 5.2 or about 10.4).

For this purpose, dispersions of the invention with different ratios M were incorporated into the above-described, polyurethane-based coating system (amounts of Al₂O₃ nanoparticles: 1.0% by weight in each case; varying amounts of wax particles to bring about the ratio M indicated above). The best results are achieved with high M values.

The results are summarized in the table hereinafter.

| | Scratch resistance (Satra) | | | | |
|---|---|---|---|---|---|
| | 500 | 1000 | 2000 | 4000 | Taber abrasion |
| | cycles | cycles | cycles | cycles | resistance (mg) |
| M = 0.6 | 1-2 | 2-3 | 2-3 | 3 | 12.3 |
| M = 5.2 | 1 | 2 | 2 | 2-3 | 8.9 |
| M = 10.4 | 1 | 1 | 1-2 | 2 | 4.6 |

1-15. (canceled)

16. A dispersion, wherein the dispersion comprises in combination:
(a) on the one hand, particles based on at least one organic material, which comprises or consists of at least one wax in the form of wax particles, wherein the wax comprises functional groups, the functional groups being polar groups which contain heteroatoms from the group of O, N and/or S; and
(b) on the other hand, particles based on at least one inorganic material in the form of inorganic nanoparticles,
where (a) the organic particles on the one hand and (b) the inorganic nanoparticles on the other hand have a bimodal particle size distribution, wherein the ratio V of the average particle diameter of the particles (a), i.e. Ø_{particle (a)}, to the average particle diameter of the particles (b), i.e. Ø_{particle (b)}, is governed by
->1; and
where the weight-based ratio M of the particles (b) to the particles (a) varies in the range from 0.01 to 500; and
(c) at least one dispersion medium.

17. The dispersion as claimed in claim 16, wherein the ratio V of the average particle diameter of the particles (a), Ø_{particle (a)}, to the average particle diameter of the particles (b), Ø_{particle (b)}, varies in the range from 1.05 to 1000.

18. The dispersion as claimed in claim 16, wherein the organic particles (a) have a particle size in the range from 5 nm to 500 μm and wherein the inorganic particles (b) have a particle size in the range from 0.5 to 1000 nm.

19. The dispersion as claimed in claim 16, wherein the weight-based ratio M of the particles (b) to the particles (a) is at least 0.5

20. The dispersion as claimed in claim 16, wherein the weight-based ratio M of the particles (b) to the particles (a) varies in the range from 0.5 to 250.

21. The dispersion as claimed in claim 16, wherein the dispersion comprises the particles (a), based on the dispersion, in amounts of 0.01% to 30% by weight and wherein the dispersion comprises the particles (b), based on the dispersion, in amounts of 0.01% to 50% by weight.

22. The dispersion as claimed in claim 16, wherein the inorganically based material of the particles (b) is formed of at least one optionally doped inorganic oxide, hydroxide, oxide hydroxide, fluoride, apatite, borate, sulfate, phosphate, sulfide, selenide, telluride, titanate, vanadate, tungstate, bismuthate, carbonate, nitride, silicate, carbide, and/or metal, an intermetallic phase, metal salt, or else of mixtures or combinations of such compounds, or comprises said compound(s)

23. The dispersion as claimed in claim 16, wherein the inorganically based material of the particles (b) is formed of at least one optionally doped oxide, hydroxide, oxide hydroxide, apatite, fluoride, borate, selenide, telluride, titanate, vanadate, tungstate, bismuthate, sulfate, phosphate, sulfide, carbonate, nitride, silicate and/or carbide of at least one metal or semimetal or else of a metal/element, an intermetallic phase, metal salt, carbon nanotubes, carbon allotropes, or else of mixtures or combinations of such compounds, or comprises said compound(s).

24. The dispersion as claimed in claim 16, wherein the inorganically based material of the particles (b) is formed of at least one optionally doped oxide, hydroxide and/or oxide hydroxide of aluminum, silicon, zinc, titanium, cerium and/or iron, an alkaline earth metal sulfate, an alkaline earth metal phosphate, alkali metal phosphate, or transition metal phosphate or lanthanoid phosphate, an alkali metal vanadate, alkaline earth metal vanadate, transition metal vanadate or lanthanoid vanadate, a cadmium sulfide or zinc sulfide, a cadmium selenide or zinc selenide, a cadmium telluride or zinc telluride, an alkaline earth metal carbonate, an aluminum nitride or silicon nitride, an alkaline earth metal silicate, a silicon carbide, carbon allotropes, a noble metal, such as silver, platinum, palladium or gold, or else of mixtures or combinations of such compounds, or comprises said compound(s).

25. The dispersion as claimed in claim 16, wherein the inorganically based material of the particles (b) is formed of aluminum oxide, silicon dioxide, zinc oxide, cerium oxide, silver, carbon nanotubes and/or titanium dioxide, or comprises said compound(s).

26. The dispersion as claimed in claim 16, wherein the inorganically based material of the particles (b) is surface-modified.

27. The dispersion as claimed in claim 16, wherein the inorganically based material of the particles (b) is surface-modified by means of polysiloxane groups.

28. The dispersion as claimed in claim 16, wherein the organically based material of the particles (a) is formed of at least one wax or comprises said wax, wherein the wax is selected from the group of (i) natural waxes; (ii) chemically modified waxes; (iii) synthetic waxes; and also mixtures thereof.

29. The dispersion as claimed in claim 28, wherein the wax is a synthetic wax based on a polyolefin-based wax or an oxidized polyolefin.

30. The dispersion as claimed in claim 16, wherein the wax comprises, as functional groups, hydroxyl groups, polyether groups, polyalkylene oxide groups and/or carboxyl groups.

31. The dispersion as claimed in claim 16, wherein the dispersion further comprises at least one further ingredient and/or at least one additive, selected from the group of emulsifiers, wetting agents, antioxidants, stabilizers, neutralizing agents, thickeners, dispersants, organic solvents, solubilizers, and biocides, and also mixtures thereof.

32. A coating material comprising a dispersion as claimed in claim 16.

33. The coating material as claimed in claim 32, wherein the coating material is selected from the group consisting of paints, inks, plastics, foams, cosmetics, nail varnishes, adhesives and sealants.
